SEQUENCE LISTING (19 sequences)

SEQ ID NO: 1 - DNA, 265 bp, SARS-Cov2
attaaaggtttataccttcccaggtaacaaaccaaccaactttcgatctcttgtagatctgttctctaaacgaactttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaag

SEQ ID NO: 2 - DNA, 270 bp, artificial sequence
  Modified 5′UTR
attaaaggtttataccttcccaggtaacaaaccaaccaactttcgatctcttgtagatctgttctctaaacgaactttaaaatctgtgtggctgtcactcggctgccgcgttattgttctgccgggcggacacgtgacgcgtaactaattactgtcgttgacaggacacgagtaactcgtctatcttctgcaggctgcttacggtttcgtccgtgttgcagccgatcatcagcacatctaggtttcgtccgggtgtgaccgaaaggtaag

SEQ ID NO: 3 - DNA, 265 bp, artificial sequence
  Modified 5′UTR
attaaaggtttataccttcccaggtaacaaaccaaccaactttcgatctcttgtagatctgttctctaaacgaactttaaaatctgtgtggctgtcactcggctgcttgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaag

SEQ ID NO: 4 - DNA, 238 bp, Artificial Sequence
  Synthesized
attaaaaaccaactttcgatctcttgtagatctgttctctaaacgaactttaaaatctgtgtggctgtcactcggctgcttgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaag

SEQ ID NO: 5 - DNA, 250 bp, Artificial Sequence
  Synthesized
attaaaggtttataccttcccaggtaacaaaccaaccaactttctgttctctaaacgaactttaaaatctgtgtggctgtcactcggctgcttgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaag

SEQ ID NO: 6 - DNA, 250 bp, Artificial Sequence
  Synthesized
attaaaggtttataccttcccaggtaacaaaccaaccaactttcgatctcttgtagatcttttaaaatctgtgtggctgtcactcggctgcttgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaag

SEQ ID NO: 7 - DNA, 221 bp, Artificial Sequence
  Synthesized
attaaaggtttataccttcccaggtaacaaaccaaccaactttcgatctcttgtagatctgttctctaaacgaactttaaaattataattaataactaattactgtcgttgacaggacacgagtaactcgtctatcttctgcaggctgcttacggtttcgtccgtgttgcagccgatcatcagcacatctaggtttcgtccgggtgtgaccgaaaggtaag

SEQ ID NO: 8 - DNA, 246 bp, Artificial Sequence
  Synthesized
attaaaggtttataccttcccaggtaacaaaccaaccaactttcgatctcttgtagatctgttctctaaacgaactttaaaatctgtgtggctgtcactcggctgcttgcttagtgcactcacgcagctgtcgttgacaggacacgagtaactcgtctatcttctgcaggctgcttacggtttcgtccgtgttgcagccgatcatcagcacatctaggtttcgtccgggtgtgaccgaaaggtaag

SEQ ID NO: 9 - DNA, 202 bp, Artificial Sequence
  Synthesized
attaaaggtttataccttcccaggtaacaaaccaaccaactttcgatctcttgtagatctgttctctaaacgaactttaaaatctgtcgttgacaggacacgagtaactcgtctatcttctgcaggctgcttacggtttcgtccgtgttgcagccgatcatcagcacatctaggtttcgtccgggtgtgaccgaaaggtaag

SEQ ID NO: 10 - DNA, 222 bp, Artificial Sequence
  Synthesized
attaaaggtttataccttcccaggtaacaaaccaaccaactttcgatctcttgtagatctgttctctaaacgaactttaaaatctgtgtggctgtcactcggctgcttgcttagtgcactcacgcagtataattaataactaattactgtcgttgacaggacacgagtaactcgtctatcttcgcacatctaggtttcgtccgggtgtgaccgaaaggtaag

SEQ ID NO: 11 - DNA, 230 bp, Artificial Sequence
  Synthesized
attaaaggtttataccttcccaggtaacaaaccaaccaactttcgatctcttgtagatctgttctctaaacgaactttaaaatctgtgtggctgtcactcggctgcttgcttagtgcactcacgcagtataattaataactaattactgtcgttgacaggacacgagtaactcgtctatcttctgcaggctgcttacggtttcgtccgtgttgcagccgatcatcagaag

SEQ ID NO: 12 - DNA, 185 bp, Artificial Sequence
  Synthesized
attaaaggtttataccttcccaggtaacaaaccaaccaactttcgatctcttgtagatctgttctctaaacgaactttaaaatctgtgtggctgtcactcggctgcttgcttagtgcactcacgcagtataattaataactaattactgtcgttgacaggacacgagtaactcgtctatcttaag

SEQ ID NO: 13 - DNA, 149 bp, Artificial Sequence
  Synthesized
attaaaggtttataccttcccaggtaacaaaccaaccaactttcgatctcttgtagatctgttctctaaacgaactttaaaatctgtgtggctgtcactcggctgcttgcttagtgcactcacgcagtataattaataactaattactg

SEQ ID NO: 14 - DNA, 190 bp, Artificial Sequence
  Synthesized
tttaaaatctgtgtggctgtcactcggctgcttgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaag

SEQ ID NO: 15 - DNA, 83 bp, Artificial Sequence
  Synthesized
attaaaggtttataccttcccaggtaacaaaccaaccaactttcgatctcttgtagatctgttctctaaacgaactttaaaat

SEQ ID NO: 16 - DNA, 300 bp, Artificial Sequence
  Synthesized
attaaaggtttataccttcccaggtaacaaaccaaccaactttcgatctcttgtagatctgttctctaaacgaactttaaaatctgtgtggctgtcactcggctgcttgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaagttggagagccttgtccctggtttcaacgagaaaac

SEQ ID NO: 17 - DNA, 225 bp, Artificial Sequence
  Synthesized
tttaaaatctgtgtggctgtcactcggctgcttgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaagttggagagccttgtccctggtttcaacgagaaaac

SEQ ID NO: 18 - DNA, 47 bp, artificial sequence
  Moderna 5′UTR
gggaaataagagagaaaagaagagtaagaagaaatataagagccacc

SEQ ID NO: 19 - RNA, 35 nt, Mus musculus
cgcguuauuguucugccgggcggacacgugacgcg

(19) United States
(12) Patent Application Publication
(10) Pub. No.: US 2024/0043835 A1
(43) Pub. Date: Feb. 8, 2024

(54) Systems and Methods for Enhancing Gene Expression

(71) Applicant: The Board of Trustees of the Leland Stanford Junior University, Stanford, CA (US)

(72) Inventors: Maria Barna, Stanford, CA (US); Kathrin Leppek, Stanford, CA (US)

(73) Assignee: The Board of Trustees of the Leland Stanford Junior University, Stanford, CA (US)

(21) Appl. No.: 17/998,788

(22) PCT Filed: May 11, 2021
(86) PCT No.: PCT/US21/31876

§ 371 Date: Nov. 14, 2022

Related U.S. Application Data
(60) Provisional application No. 63/022,901, filed on May 11, 2020.

(51) Int. Cl. C12N 15/11

(52) U.S. Cl. CPC C12N 15/11

(57) ABSTRACT

Systems, methods, and kits for enhancing mRNA translation are disclosed. Some embodiments describe expression constructs for producing a peptide and include a translational enhancer. Additional embodiments describe methods for producing a peptide using a construct including a translational enhancer. Certain embodiments further enhance mRNA stability.

### CROSS-REFERENCE TO RELATED APPLICATIONS

The current application claims priority to U.S. Provisional Patent Application No. 63/022,901, entitled “Systems and Methods for Enhancing Gene Expression” to Maria Barna et al., filed May 11, 2020, the disclosure of which is hereby incorporated by reference in its entirety.

### STATEMENT REGARDING FEDERALLY SPONSORED RESEARCH OR DEVELOPMENT

This invention was made with Governmental support under Contract No. 1R01HD086634 awarded by the National Institutes of Health. The government has certain rights in the invention.

### FIELD OF THE DISCLOSURE

The present disclosure relates to gene regulation, and in particular methods, systems, and compositions enhance gene expression.

### INCORPORATION OF SEQUENCE LISTING

A computer readable form of the sequence listing, “06771 PCT_Sequences_ST25.txt”, submitted via EFS-WEB, is herein incorporated by reference in its entirety.

### BACKGROUND OF THE DISCLOSURE

Messenger RNA (mRNA) based therapeutics hold the potential to transform modern medicine because of their fast production and use for precise therapies involving reprogramming patients' own cells to produce therapeutic proteins. Compared to the development of recombinant proteins, production of mRNA is faster, more cost-effective, and more flexible because it can be easily produced by in vitro transcription. However, technical obstacles facing mRNA pharmaceuticals are also apparent. These obstacles include the optimization of the stability, translation efficiency, and delivery mechanisms for RNA therapeutics, which are all pivotal issues that need to be carefully optimized for preclinical and clinical applications. For example, mRNA vaccines still suffer from decreased efficacy due to poor expression of the payload m RNA. Poor expression creates an obstacle to dosing of mRNA-based therapeutics that has not been resolved.

### SUMMARY OF THE DISCLOSURE

This summary is meant to provide examples and is not intended to be limiting of the scope of the invention in any way. For example, any feature included in an example of this summary is not required by the claims, unless the claims explicitly recite the feature. Also, the features described can be combined in a variety of ways. Various features and steps as described elsewhere in this disclosure can be included in the examples summarized here.

Systems and methods for enhancing gene expression are disclosed. In one embodiment, a construct to enhance gene translation includes a coding region and a 5′-UTR located at the 5′ end of the coding region and including at least one translational enhancer.

In a further embodiment, the 5′-UTR further includes a spacer located between the translational enhancer and the coding region.

In another embodiment, the spacer is approximately 35-150 nt in length.

In a still further embodiment, the translational enhancer is a SARS-CoV2 5′-UTR.

In still another embodiment, the translational enhancer is sequence variant of a SARS-CoV2 5′-UTR.

In a yet further embodiment, the translational enhancer is SEQ ID NO: 1 or a sequence variant thereof.

In yet another embodiment, the translational enhancer is selected from SEQ ID NOs: 1-17.

In a further embodiment again, a method for producing a peptide includes obtaining an expression construct possessing a target gene and a 5′-UTR, where the expression construct includes a coding region and a 5′-UTR located at the 5′ end of the coding region and including at least one translational enhancer, and delivering the expression construct to a ribosome for translation.

In another embodiment again, the 5′-UTR further includes a spacer located between the translational enhancer and the coding region.

In a further additional embodiment, the spacer is approximately 35-150 nt in length.

In another additional embodiment, the translational enhancer is a SARS-CoV2 5′-UTR.

In a still yet further embodiment, the translational enhancer is sequence variant of a SARS-CoV2 5′-UTR.

In still yet another embodiment, the translational enhancer is SEQ ID NO: 1 or a sequence variant thereof.

In a still further embodiment again, the translational enhancer is selected from SEQ ID NOs: 1-17.

In still another embodiment again, the method further includes isolating a peptide produced by the ribosome using the expression cassette.

In a still further additional embodiment, a medical formulation includes an RNA molecule including a coding region and a 5′-UTR located at the 5′ end of the coding region and including at least one translational enhancer.

In still another additional embodiment, the medical formulation further includes one or more of a buffer, a lubricant, a binder, a flavorant, and a coating.

In a yet further embodiment again, the formulation is delivered to an individual orally, nasally, inhalationally, parentally, intravenously, intraperitoneally, subcutaneously, intramuscularly, intradermally, topically, rectally, intracerebrally, intraventricularly, intracerebroventricularly, intrathecally, intracisternally, intraspinally, perispinally, intraocularly, or intravitreally.

In yet another embodiment again, the 5′-UTR further includes a spacer located between the translational enhancer and the coding region.

In a yet further additional embodiment, the spacer is approximately 100-150 nt in length.

In yet another additional embodiment, the translational enhancer is a SARS-CoV2 5′-UTR.

In a further additional embodiment again, the translational enhancer is sequence variant of a SARS-CoV2 5′-UTR.

In another additional embodiment again, the translational enhancer is SEQ ID NO: 1 or a sequence variant thereof.

In a still yet further embodiment again, the translational enhancer is selected from SEQ ID NOs: 1-17.

The foregoing and other objects, features, and advantages of the disclosed technology will become more apparent from the following detailed description, which proceeds with reference to the accompanying figures.

### BRIEF DESCRIPTION OF THE DRAWINGS

FIGS. 1A-1B illustrate a structure of a SARS-CoV2 5′ untranslated region (UTR) in accordance with various embodiments of the invention.

FIGS. 2A-2B illustrate exemplary polysome fractionation data in accordance with various embodiments of the invention.

FIG. 3 illustrates an expression construct in accordance with various embodiments of the invention.

FIG. 4 illustrates a method for producing a peptide or protein in accordance with various embodiments of the invention.

### DETAILED DESCRIPTION OF THE DISCLOSURE

The mechanisms of eukaryotic translation initiation and principles of its regulation are of great interest both with respect to new layers of control to gene expression as well as for the discovery of novel sequences and structures that can boost the translation of downstream open reading frames. Such translation regulatory regions can be extended to the design of RNA vaccines, viral-based therapies, as well as the production of any protein in cells and organism. Translation involves an interaction between mRNA, which code for certain proteins or peptides, and ribosomes, which assemble a peptide from the mRNA sequence.

Certain organisms, including viruses, have evolved or adapted to include sequences on their RNA for post-transcriptional regulation. Additionally, some of these sequences introduce secondary structures, such as stem-loop structures, which increase overall stability of an mRNA, granting a longer life for RNA within a cell.

Turning now to the drawings and data, embodiments herein are directed to methods, systems, and compositions to enhance gene expression. Many embodiments introduce sequences to DNA or RNA, including mRNA, which increase translation and/or stability of an mRNA. Certain embodiments are capable of cap-dependent and/or cap-independent translation.

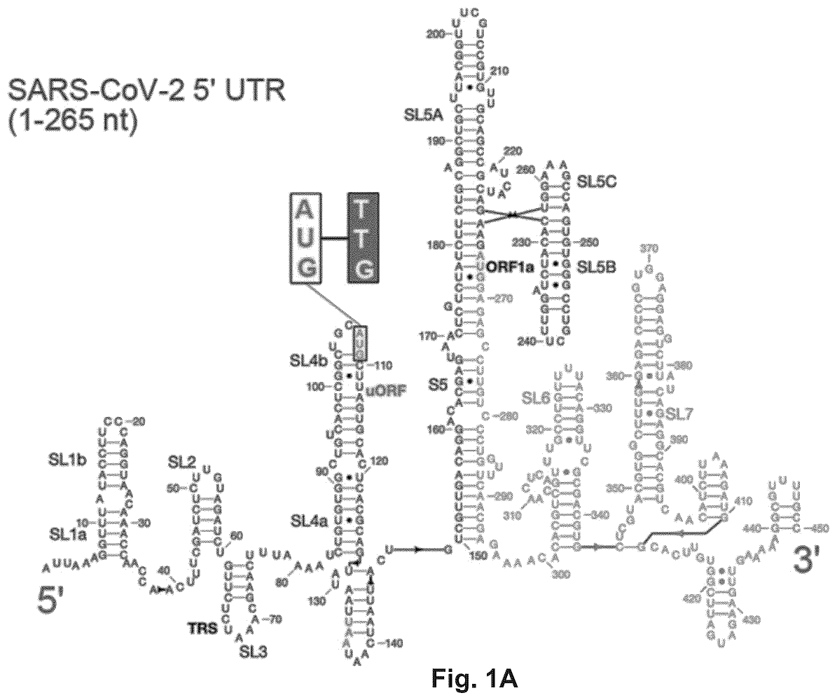

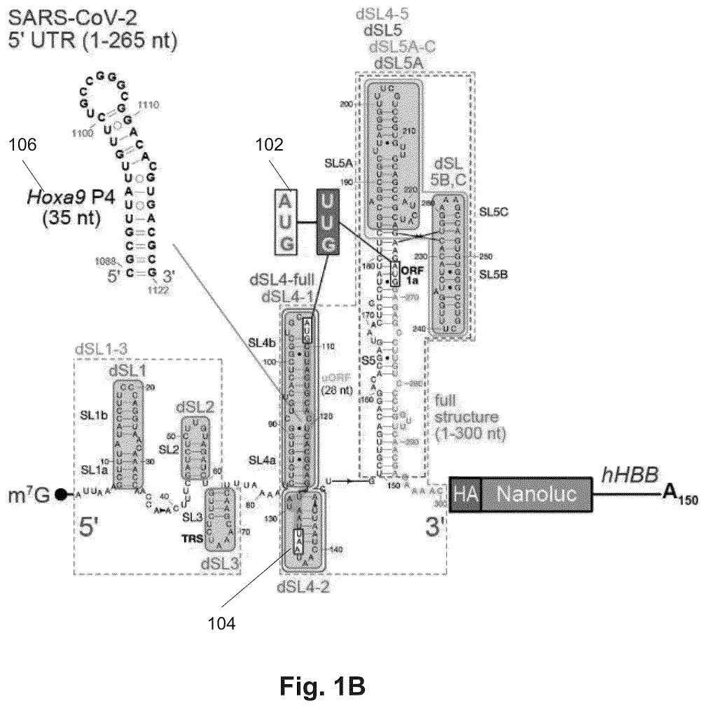

Turning to FIG. 1A, a structure of a SARS-CoV2 5′ untranslated region (5′-UTR) (SEQ ID NO: 1) is illustrated attached to an open reading frame. Many embodiments are directed to using SARS-CoV2 5′-UTR (SEQ ID NO: 1) and/or variants thereof (SEQ ID NOs: 2-3) to enhance translation and/or stability of mRNAs. The SARS-CoV2 5′-UTR (black letters) is a 265 nt length sequence exhibiting multiple stem-loop structures. As noted in FIG. 1, SARS-CoV2 5′-UTR possesses an AUG located at nucleotides 107-09 and a UAA at nucleotides 134-36 of the 5′-UTR. AUG represents the canonical start codon for translation, while UAA represents a canonical stop codon for translation, indicating that the SARS-CoV2 5′-UTR may contain a pseudo or unknown open reading frame (ORF) within its structure. Certain embodiments alter the sequence of SARS-CoV2 5′-UTR to identify additional improvements to stability and/or translation. In certain embodiments, this apparent ORF of the SARS-CoV2 5′-UTR is replaced is replaced with a P4 element from the homeobox a9 gene (Hoxa9) 5′-UTR. In these embodiments, the SARS-CoV2 5′-UTR is termed CoV2 5′-UTR-P4 (SEQ ID NO: 2). Additional embodiments alter the AUG at nucleotides 107-09 to TTG, also noted as CoV2 5′-UTR (AUG→TTG) (SEQ ID NO: 3).

Turning to FIG. 1B, some embodiments are directed to structural variants, deletion variants, and/or truncations of SARS-CoV2 5′-UTR (e.g., SEQ ID NOs: 4-17). Specifically, FIG. 1B illustrates the 265 nt of a full-length SARS-CoV2 5′-UTR (SEQ ID NOs: 1) attached to a reporter gene construct including a reporter gene and human β-globin 3′-UTR sequence. In certain embodiments, deletion variants remove one or more stem-loop structures within the full-length SARS-CoV2 5′-UTR. FIG. 1B illustrates various stem-loop structures that are deleted from a full-length SARS-CoV2 5′-UTR in accordance with various embodiments, where the various, deleted stem-loop structures are labeled (e.g., dSL1, dSL1-3, etc.) and boxed (solid and/or dashed) in accordance with certain embodiments. Table 1 provides a list of exemplary deletion embodiments and its coordinated SEQ ID NO. For example, a dSL1 variant comprises the SARS-CoV2 5′-UTR without the boxed stem-loop structure identified by dSL1, while dSL1-3 variant comprises the SARS-CoV2 5′-UTR without the boxed stem-loop structure identified by dSL1-3.

As noted herein, a full-length SARS-CoV2 5′-UTR contains canonical start codon sequences “AUG” within its sequence. In some embodiments, these sequences have been altered to prevent possible premature translation, as noted at 102. Additionally, 104 identifies a canonical stop codon located with a full-length SARS-CoV2 5′-UTR, which creates a possible open reading frame ORF within the UTR. In some embodiments, this ORF is replaced with another sequence, such as a P4 sequence 106 (SEQ ID NO: 19).

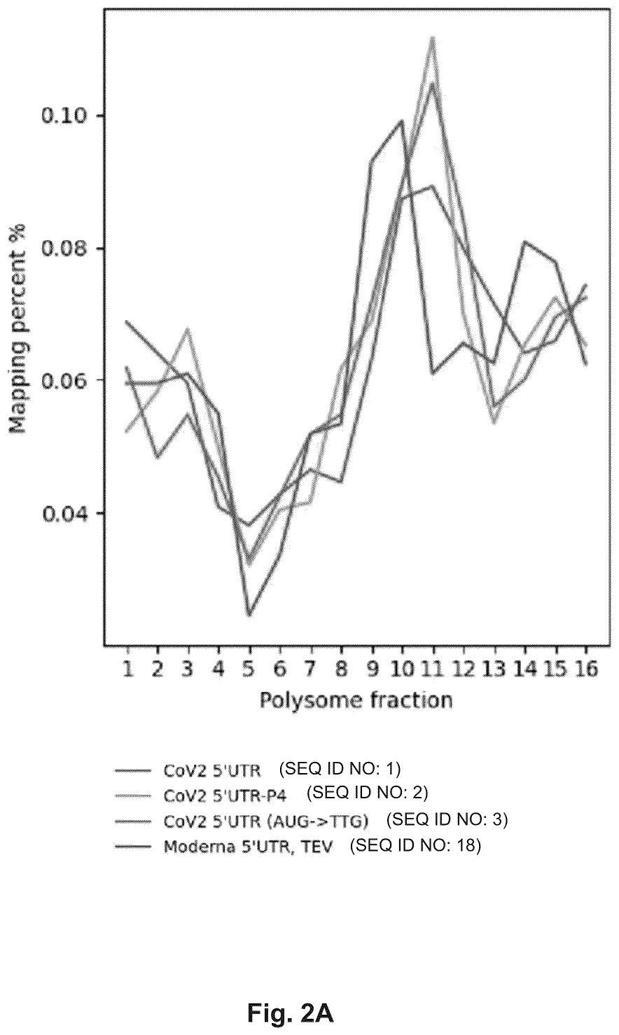

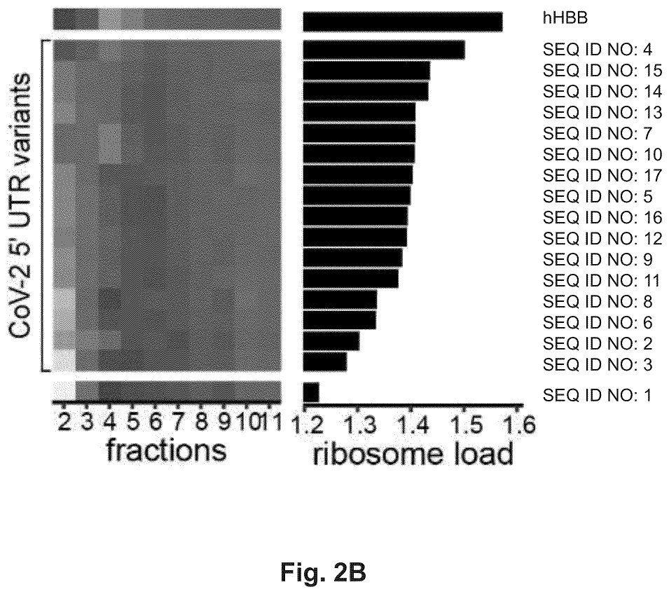

Turning to FIG. 2A, polysome fractionation data in accordance with various embodiments is illustrated. Polysome fractionation illustrates a number of ribosomes attached to a particular mRNA, where increased numbers of ribosomes (e.g., higher polysome fractions) correlate to higher levels of translation. FIG. 2 shows a comparison of SARS-CoV2 5′-UTR (SEQ ID NO: 1), SARS-CoV2 5′-UTR-P4 (SEQ ID NO: 2), and SARS-CoV2 5′-UTR (AUG→TTG) (SEQ ID NO: 3) as compared to a reference 5′-UTR (SEQ ID NO: 4). As illustrated, a higher percent of mRNAs from SARS-CoV2 5′-UTR-P4 (SEQ ID NO: 2) and SARS-CoV2 5′-UTR (AUG→TTG) (SEQ ID NO: 3) are above the reference 5′-UTR (SEQ ID NO: 4), indicating an improvement of certain embodiments over the reference 5′-UTR (SEQ ID NO: 4).

Similarly, FIG. 2B illustrates a heatmap of polysome fraction and ribosomal load of various embodiments, including a full-length SARS-CoV2 5′-UTR (SEQ ID NO: 1), SARS-CoV2 5′-UTR-P4 (SEQ ID NO: 2), and SARS-CoV2 5′-UTR (AUG→TTG) (SEQ ID NO: 3), and additional deletion variants (SEQ ID NOs: 4-17) as compared to a β-globin 5′-UTR (hHBB) as a standard.

### Constructs for Enhancing Translation

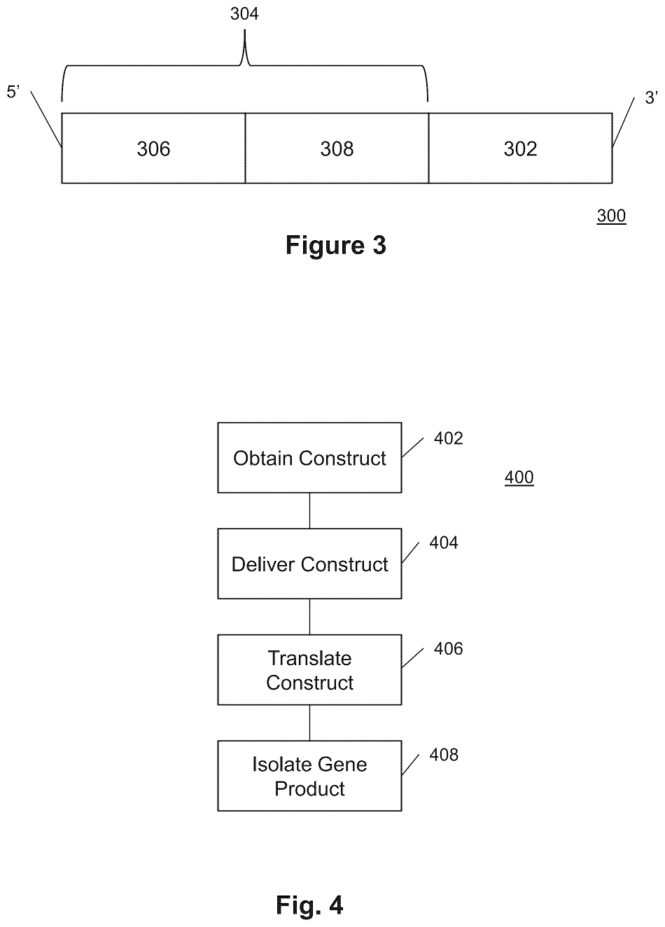

Turning FIG. 3, Many embodiments are directed to expression constructs 300 incorporating translational enhancers. Constructs of numerous embodiments include a coding region 302 and a 5′-UTR 304 located at the 5′ end of coding region 302. In certain embodiments, the coding region 302 is selected for increased production of its resultant protein or peptide and can include a particular gene. In some embodiments, a gene is a natural gene isolated from an organism or species, while certain embodiments the gene is an artificial or designed gene to generate a specific peptide. In many embodiments a 5′-UTR 304 includes a translational enhancer 306. In many embodiments, the translational enhancer 306 is a SARS-CoV 5′-UTR (SEQ ID NO: 1). While additional embodiments are modifications of the SARS-CoV 5′-UTR (SEQ ID NOs: 2-17), such as sequence modifications, deletion variants, structural variants, and/or truncations.

In many embodiments, the 5′-UTR 304 further comprises a spacer 308 located between coding region 302 and translational enhancer 306. In many embodiments, spacer 308 is approximately 35-150 nt in length. In many embodiments, an expression construct 300 is made of RNA, such that the construct is translated into a protein or peptide. In some embodiments, an expression construct 300 is made of DNA along with at least one of a promoter, an enhancer, transcription start site, and/or any other components to transcribe DNA to RNA. Additional embodiments include one or more additional features, such as a 5′ cap, a spacer region, 3′ tail, and/or any other features that assist with translation. It should be noted that while certain sequences within SEQ ID NOs: 1-19 are listed as either DNA or RNA, one of skill in the art would understand how to create an RNA construct from a DNA sequence and/or a DNA construct from an RNA sequence, depending on specific need or use for a specific purpose.

### Methods of Producing a Protein or Peptide

FIG. 4 illustrates a method 400 for producing a protein or peptide. Many embodiments obtain an expression construct at 402. Expression constructs are described elsewhere herein and can be DNA, where the construct is transcribed to mRNA for translation, while some embodiments obtain the construct as RNA, which can be imminently translated.

In various embodiments, an expression construct is delivered to a ribosome for translation 404. In some embodiments, the expression construct is delivered to a cell for translation within the cell (e.g., transfection), such as for production of a peptide and/or protein. Certain embodiments mix the construct to a solution including ribosomes, such as cellular lysate, for in vitro expression. Further embodiments deliver the construct to a mammal or other organism for treatment, including (but not limited to) for purposes of introducing viral-based therapies, (e.g., RNA vaccines) or production of a protein or peptide (e.g., gene therapy to replace or supplement innate proteins and/or peptides). In certain embodiments, the construct is encapsulated in a larger structure for delivery and/or incorporation into a cell, such as a capsid, lipid nanoparticle, micelle, bacterium, extracellular vesicle, and/or any other means for delivering the construct. In certain embodiments, delivery is accomplished via microinjection, particle bombardment, or other direct means. In certain embodiments involving the treatment of an individual, an RNA construct can be formulated for a medical use, including by combining it with one or more buffers, lubricants, binders, flavorants, and coatings. Various embodiments an expression construct for specific transfection, such as through a virus (e.g., adeno-associated viruses (AAVs)), viroids, capsids, micelles, and/or larger DNA and/or RNA structures suitable for targeting and/or stability. Various embodiments delivery medical formulations to an individual via one or more paths selected from orally, nasally, inhalationally, parentally, intravenously, intraperitoneally, subcutaneously, intramuscularly, intradermally, topically, rectally, intracerebrally, intraventricularly, intracerebroventricularly, intrathecally, intracisternally, intraspinally, perispinally, intraocularly, intravitreally, and/or any other means to deliver an expression construct most effectively to a tissue, cell, and/or organ being treated.

In some embodiments, the construct is translated 406 to produce a protein or peptide. In various embodiments, translation is accomplished by incubating a culture or reaction tube at an appropriate temperature. In additional embodiments, including where a construct is delivered to an organism, the reaction is allowed to proceed with little monitoring or incubation.

At 408, many embodiments isolate a gene product (e.g., protein or peptide) of the construct. Certain embodiments isolate the gene product by various means, including chromatographic methods, such as size-exclusion and/or ion-exchange chromatography, pulldown methods, and/or other means of isolating a protein from solution.

### Kits for Gene Expression

Certain embodiments are directed to kits to increase gene expression and/or mRNA translation in an organism. Such embodiments include at least one nucleic acid (either RNA or DNA) with a 5′-UTR sequence (e.g., 5′-UTR 204, FIG. 2). In some embodiments, the 5′-UTR is joined to a target gene sequence (e.g., target gene 202, FIG. 2) via ligation, PCR, and/or a combination thereof. In ligation, certain embodiments include an adapter sequence located at the 3′ end of the 5′-UTR, to allow for a complementary sequence to anneal to the adapter sequence. Other embodiments utilize blunt end ligation, especially in single stranded molecules (e.g., RNA), where complementary sequences are not possible. Embodiments employing ligation further include one or more enzymes (e.g., ligases, topoisomerases, etc.) to ligate the ends of the 5′-UTR and the target gene. Further embodiments alter one or more end of the 5′-UTR and/or the target gene to prevent aberrant ligation between a 5′-UTR and target gene. In some of these embodiments, the 5′-UTR includes a blocking modification on the 5′ end of the 5′-UTR to prevent ligation on the 5′ end. Additional embodiments include enzymes and other reagents to modify the target gene by removing and/or adding phosphate groups and hydroxy groups to the 5′ and/or 3′ ends of the target gene to increase appropriate ligation.

In embodiments employing PCR to add a 5′-UTR to a target gene, the 5′-UTR includes a primer sequence for amplification of a target sequence. The primer sequence can be gene-specific primer. Further embodiments employ a universal primer, such that the primer sequence amplifies the target gene regardless of the target gene sequence. In certain embodiments, a universal primer is concatenated to a gene-specific primer sequence. In such embodiments, two PCR reactions can be employed where the first PCR reaction adds the universal primer to the target gene sequence, while the second PCR adds the 5′-UTR onto the universal primer. PCR-based embodiments include enzymes and reagents for a PCR reaction, including NTPs, dNTPs, buffer, and one or more polymerases, as necessary for amplification of a nucleic acid sequences.

Embodiments employing both PCR and ligation may ligate a universal primer on to target gene sequences, followed by amplification to add the 5′-UTR to the target gene sequence.

Further embodiments include components for transfection or introduction of an expression construct (e.g., 5′-UTR—gene construct). Some embodiments include a plasmid or other larger construct for preservation, replication, and/or transfection of the expression construct. Further embodiments include a delivery mechanism for delivering the construct to a cell or organism. Delivery mechanisms in accordance with various embodiments include bacterial vectors, viral vectors, particle bombardment, other means for introducing the expression construct, and combinations thereof.

### DOCTRINE OF EQUIVALENTS

Having described several embodiments, it will be recognized by those skilled in the art that various modifications, alternative constructions, and equivalents may be used without departing from the spirit of the invention. Additionally, a number of well-known processes and elements have not been described in order to avoid unnecessarily obscuring the present invention. Accordingly, the above description should not be taken as limiting the scope of the invention.

Those skilled in the art will appreciate that the foregoing examples and descriptions of various preferred embodiments of the present invention are merely illustrative of the invention as a whole, and that variations in the components or steps of the present invention may be made within the spirit and scope of the invention. Accordingly, the present invention is not limited to the specific embodiments described herein, but, rather, is defined by the scope of the appended claims.

TABLE 1
Deletion Variants of SARS-CoV-2 5′-UTR

| Sequence Name | SEQ ID NO: |
|---|---|
| TTG-dSL1 | 4 |
| TTG-dSL2 | 5 |
| TTG-dSL3 | 6 |
| TTG-dSL4-1 | 7 |
| TTG-dSL4-2 | 8 |
| TTG-dSL4-full | 9 |
| TTG-dSL5A | 10 |
| TTG-dSL5B,C | 11 |
| TTG-dSL5A-C | 12 |
| TTG-dSL5 | 13 |
| TTG-dSL1-3 | 14 |
| TTG-dSL4-5 | 15 |
| TTG-TTGfull | 16 |
| TTG-TTGfull-dSL1-3 | 17 |

1. A construct to enhance gene translation, comprising:
a coding region; and
a 5′-UTR located at the 5′ end of the coding region and comprising at least one translational enhancer.

2. The construct of claim 1, wherein the 5′-UTR further comprises a spacer located between the translational enhancer and the coding region.

3. The construct of claim 2, wherein the spacer is approximately 35-150 nt in length.

4. The construct of claim 1, wherein the translational enhancer is a SARS-CoV2 5′-UTR or a sequence variant thereof.

5. (canceled)

6. The construct of claim 1, wherein the translational enhancer is SEQ ID NO: 1 or a sequence variant thereof.

7. The construct of claim 1, wherein the translational enhancer is selected from SEQ ID NOs: 1-17.

8. A method for producing a peptide, comprising:
obtaining an expression construct possessing a target gene and a 5′-UTR, wherein the expression construct comprises a coding region and a 5′-UTR located at the 5′ end of the coding region and comprising at least one translational enhancer; and
delivering the expression construct to a ribosome for translation.

9. The method of claim 8, wherein the 5′-UTR further comprises a spacer located between the translational enhancer and the coding region.

10. The method of claim 9, wherein the spacer is approximately 35-150 nt in length.

11. The method of claim 8, wherein the translational enhancer is a SARS-CoV2 5′-UTR or a sequence variant thereof.

12. (canceled)

13. The method of claim 8, wherein the translational enhancer is SEQ ID NO: 1 or a sequence variant thereof.

14. The method of claim 8, wherein the translational enhancer is selected from SEQ ID NOs: 1-17.

15. The method of claim 8, further comprising isolating a peptide produced by the ribosome using the expression cassette.

16. A medical formulation comprising:
an RNA molecule comprising:
a coding region; and
a 5′-UTR located at the 5′ end of the coding region and comprising at least one translational enhancer.

17. The medical formulation of claim 16, further comprising one or more of a buffer, a lubricant, a binder, a flavorant, and a coating.

18. The medical formulation of claim 16, wherein the formulation is delivered to an individual orally, nasally, inhalationally, parentally, intravenously, intraperitoneally, subcutaneously, intramuscularly, intradermally, topically, rectally, intracerebrally, intraventricularly, intracerebroventricularly, intrathecally, intracisternally, intraspinally, perispinally, intraocularly, or intravitreally.

19. The medical formulation of claim 16, wherein the 5′-UTR further comprises a spacer located between the translational enhancer and the coding region.

20. (canceled)

21. The construct of claim 16, wherein the translational enhancer is a SARS-CoV2 5′-UTR or a sequence variant thereof.

22. (canceled)

23. The construct of claim 16, wherein the translational enhancer is SEQ ID NO: 1 or a sequence variant thereof.

24. The construct of claim 16, wherein the translational enhancer is selected from SEQ ID NOs: 1-17.
